(19) United States
(12) Patent Application Publication
(10) Pub. No.: US 2025/0114065 A1
(43) Pub. Date: Apr. 10, 2025

(54) SYSTEM AND METHOD FOR EVALUATING MUSCLE QUALITY USING ULTRASOUND

(71) Applicant: BODYMAP INC, Gyeonggi-do (KR)

(72) Inventor: Sung Yun PARK, Seoul (KR)

(73) Assignee: BODYMAP INC, Gyeonggi-do (KR)

(21) Appl. No.: 18/730,633

(22) PCT Filed: Jan. 11, 2023
(86) PCT No.: PCT/KR2023/000480

§ 371 Date: Jul. 19, 2024

(30) Foreign Application Priority Data
Jan. 19, 2022 (KR) 10-2022-0007853

(51) Int. Cl. A61B 8/08; A61B 8/00; G01S 7/52

(52) U.S. Cl. CPC A61B 8/08; G01S 7/52036; A61B 8/461

(57) ABSTRACT

The present invention relates to a system and method for evaluating muscle quality using ultrasound, and more specifically, to a system and method for measuring an ultrasound signal of a subject using a low-cost A-mode ultrasonic probe, and evaluating muscle quality on the basis thereof.

According to one embodiment of the present invention, the advantage of reducing the cost required for muscle quality evaluation is provided, while also providing the same accuracy as muscle quality evaluation using a B-mode ultrasound device.

### TECHNICAL FIELD

The present disclosure relates to a system and method for evaluating muscle quality using ultrasound, and particularly, to a system and method for measuring an ultrasound signal of a subject by using a cheap A-mode ultrasonic probe and evaluating muscle quality based on the ultrasound signal.

### BACKGROUND ART

FIG. 1 is a diagram for describing muscle quality according to an embodiment of the present disclosure.

Referring to FIG. 1, muscle quality according to the present disclosure means a degree of pimelosis of muscles. A phenomenon in which fat builds up on muscles, like a fatty liver in which fat builds up on liver, is called myosteatosis (or muscles pimelosis). As muscles pimelosis is too far along, muscle quality is reduced. FIG. 1(a) illustrates the state in which muscle quality is relatively low. FIG. 1(b) illustrates the state in which muscle quality is relatively high.

There is a need to develop a system capable of measuring and evaluating muscle quality, as an index for checking an individual's metabolic health, based on the results of research in which there is a high probability that a person who has more muscles having better quality will be metabolically healthy due to a lower risk of diabetes, high blood pressure, etc.

Ultrasound means a sound wave having a high frequency of an audible frequency range or more, which can be heard by a person. Ultrasonography means that a signal that is reflected after the human body transmits a pulse wave in tissues having an acoustic impedance difference is amplified, converted, and then indicated as in the form of an image through a computer.

An ultrasonography apparatus refers to an apparatus that images a reflected signal that is obtained by transmitting and receiving ultrasound to and from a target portion of a body to be inspected, and is divided into an amplitude (A)-mode, a brightness (B)-mode, a motion (M)-mode, and a doppler (D)-mode depending on an image display method.

An image display method in the B-mode has been basically used in a conventional ultrasonography apparatus because the A-mode has lower accuracy than the B-mode.

However, a B-mode ultrasonography apparatus essentially requires a component for outputting an image. Such an image output unit is often expensive equipment. For this reason, there is a problem in that in order to analyze the state of muscles of a human body by using ultrasound, an examinee has to purchase expensive equipment or has to visit a hospital by which expensive equipment can be relatively easily purchased so as to diagnose his or her muscle state using ultrasound.

### DISCLOSURE

### Technical Problem

In order to solve the aforementioned problem, an object of the present disclosure is to provide a system and method for evaluating muscle quality using ultrasound, which can evaluate muscle quality by using an A-mode ultrasound apparatus.

### Technical Solution

As an embodiment of the present disclosure, there is provided a system for evaluating muscle quality using ultrasound.

The system for evaluating muscle quality using ultrasound according to an embodiment of the present disclosure may include an ultrasound transceiver configured to transmit ultrasound to a subject and to receive a plurality of reflected sounds that are reflected by the subject, a signal processor configured to implement a reflected signal by using the plurality of reflected sounds, a peak extractor configured to extract peaks from the reflected signal, and an analyzer configured to calculate a ratio of materials that constitute the subject by using the peaks and to evaluate muscle quality based on the calculated ratio.

In the system for evaluating muscle quality using ultrasound according to an embodiment of the present disclosure, the ultrasound transceiver may be at least one in number.

In the system for evaluating muscle quality using ultrasound according to an embodiment of the present disclosure, the signal processor may implement the reflected signal by displaying strength of the plurality of reflected sounds in a time axis in the form of an amplitude size.

In the system for evaluating muscle quality using ultrasound according to an embodiment of the present disclosure, the signal processor may further include a filtering unit configured to perform filtering processing on the reflected signal and a smoothing processing unit configured to perform smoothing processing on the reflected signal.

In the system for evaluating muscle quality using ultrasound according to an embodiment of the present disclosure, the filtering unit may remove a contact-reflected signal implemented by a reflected sound that is generated at the interface of the ultrasound transceiver and the subject.

In the system for evaluating muscle quality using ultrasound according to an embodiment of the present disclosure, the material may include muscles, a bone, or fat.

In the system for evaluating muscle quality using ultrasound according to an embodiment of the present disclosure, the analyzer may extract an interval between a plurality of peaks included in the reflected signal and calculate the thickness of fat or muscles based on the interval.

In the system for evaluating muscle quality using ultrasound according to an embodiment of the present disclosure, the analyzer may further include a Fourier transform unit configured to perform short time Fourier transform (STFT) processing on the reflected signal and a pimelosis degree calculation unit configured to calculate a degree of pimelosis of muscles by analyzing the reflected signal on which the STFT processing has been performed.

As an embodiment of the present disclosure, there is provided a method of evaluating muscle quality using ultrasound.

The method of evaluating muscle quality using ultrasound according to an embodiment of the present disclosure may include an ultrasound transmission and reception step of transmitting, by an ultrasound transceiver, ultrasound to a subject and receiving a plurality of reflected sounds that are reflected by the subject, a signal processing step of implementing, by a signal processor, a reflected signal by using the plurality of reflected sounds, a peak extraction step of extracting, by a peak extractor, peaks from the reflected signal, and an analysis step of calculating, by an analyzer, a ratio of materials that constitute the subject by using the peaks and evaluating muscle quality based on the calculated ratio.

In the method of evaluating muscle quality using ultrasound according to an embodiment of the present disclosure, the ultrasound transceiver may be at least one in number.

In the method of evaluating muscle quality using ultrasound according to an embodiment of the present disclosure, the reflected signal may be implemented by displaying strength of a plurality of reflected sounds in a time axis in the form of an amplitude size.

In the method of evaluating muscle quality using ultrasound according to an embodiment of the present disclosure, the signal processing step may further include a filtering f performing, by a filtering unit, filtering processing on the reflected signal and a smoothing processing step of performing, by a smoothing processing unit, smoothing processing on the reflected signal.

In the method of evaluating muscle quality using ultrasound according to an embodiment of the present disclosure, the filtering step may include removing a contact-reflected signal implemented by a reflected sound that is generated at the interface of the ultrasound transceiver and the subject.

In the method of evaluating muscle quality using ultrasound according to an embodiment of the present disclosure, the material may include muscles, a bone, or fat.

In the method of evaluating muscle quality using ultrasound according to an embodiment of the present disclosure, the analysis step further may include steps of extracting an interval between a plurality of peaks included in the reflected signal and calculating the thickness of fat or muscles based on the interval.

In the method of evaluating muscle quality using ultrasound according to an embodiment of the present disclosure, the analysis step may further include a Fourier transform step of performing, by a Fourier transform unit, short time Fourier transform (STFT) processing on the reflected signal and a pimelosis degree calculation step of calculating, by a pimelosis degree calculation unit, a degree of pimelosis of muscles by analyzing the reflected signal on which the STFT processing has been performed.

As an embodiment of the present disclosure, there is provided a computer-readable recording medium on which a program for implementing the aforementioned method has been recorded.

### Advantageous Effects

According to an embodiment of the present disclosure, there are advantages in that the same accuracy as that of the evaluation of muscle quality using a B-mode ultrasound apparatus is achieved and a cost for evaluating muscle quality is reduced.

Effects of the present disclosure which may be obtained in the present disclosure are not limited to the aforementioned effects, and the other effects not described above may be evidently understood by a person having ordinary knowledge in the art to which the present disclosure pertains from the following description.

### BRIEF DESCRIPTION OF DRAWINGS

FIG. 1 is a diagram for describing muscle quality according to an embodiment of the present disclosure.

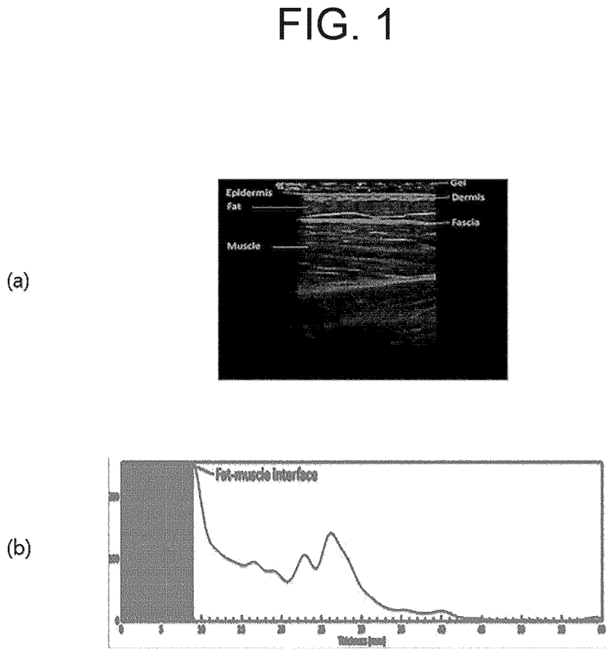

FIG. 2 illustrates a difference between image display methods of a B-mode ultrasonography apparatus and an A-mode ultrasonography apparatus.

FIG. 3 is a block diagram of a system for evaluating muscle quality using ultrasound according to an embodiment of the present disclosure.

FIG. 4 is a block diagram of a signal processor 200 according to an embodiment of the present disclosure.

FIG. 5 is a diagram of a signal processing process of the signal processor 200 according to an embodiment of the present disclosure.

FIG. 6 is a diagram of a muscle quality evaluation process of an analyzer 400 according to an embodiment of the present disclosure.

FIG. 7 is a block diagram of the analyzer 400 according to another embodiment of the present disclosure.

FIG. 8 is a diagram of an STFT process of a Fourier transform unit 410 according to another embodiment of the present disclosure.

FIG. 9 is a flowchart of a method of evaluating muscle quality using ultrasound according to an embodiment of the present disclosure.

### BEST MODE

As an embodiment of the present disclosure, there is provided a system for evaluating muscle quality using ultrasound.

The system for evaluating muscle quality using ultrasound according to an embodiment of the present disclosure may include an ultrasound transceiver configured to transmit ultrasound to a subject and to receive a plurality of reflected sounds that are reflected by the subject, a signal processor configured to implement a reflected signal by using the plurality of reflected sounds, a peak extractor configured to extract peaks from the reflected signal, and an analyzer configured to calculate a ratio of materials that constitute the subject by using the peaks and to evaluate muscle quality based on the calculated ratio.

In the system for evaluating muscle quality using ultrasound according to an embodiment of the present disclosure, the ultrasound transceiver may be at least one in number.

In the system for evaluating muscle quality using ultrasound according to an embodiment of the present disclosure, the signal processor may implement the reflected signal by displaying strength of the plurality of reflected sounds in a time axis in the form of an amplitude size.

In the system for evaluating muscle quality using ultrasound according to an embodiment of the present disclosure, the signal processor may further include a filtering unit configured to perform filtering processing on the reflected signal and a smoothing processing unit configured to perform smoothing processing on the reflected signal.

In the system for evaluating muscle quality using ultrasound according to an embodiment of the present disclosure, the filtering unit may remove a contact-reflected signal implemented by a reflected sound that is generated at the interface of the ultrasound transceiver and the subject.

In the system for evaluating muscle quality using ultrasound according to an embodiment of the present disclosure, the material may include muscles, a bone, or fat.

In the system for evaluating muscle quality using ultrasound according to an embodiment of the present disclosure, the analyzer may extract an interval between a plurality of peaks included in the reflected signal and calculate the thickness of fat or muscles based on the interval.

In the system for evaluating muscle quality using ultrasound according to an embodiment of the present disclosure, the analyzer may further include a Fourier transform unit configured to perform short time Fourier transform (STFT) processing on the reflected signal and a pimelosis degree calculation unit configured to calculate a degree of pimelosis of muscles by analyzing the reflected signal on which the STFT processing has been performed.

As an embodiment of the present disclosure, there is provided a method of evaluating muscle quality using ultrasound.

The method of evaluating muscle quality using ultrasound according to an embodiment of the present disclosure may include an ultrasound transmission and reception step of transmitting, by an ultrasound transceiver, ultrasound to a subject and receiving a plurality of reflected sounds that are reflected by the subject, a signal processing step of implementing, by a signal processor, a reflected signal by using the plurality of reflected sounds, a peak extraction step of extracting, by a peak extractor, peaks from the reflected signal, and an analysis step of calculating, by an analyzer, a ratio of materials that constitute the subject by using the peaks and evaluating muscle quality based on the calculated ratio.

In the method of evaluating muscle quality using ultrasound according to an embodiment of the present disclosure, the ultrasound transceiver may be at least one in number.

In the method of evaluating muscle quality using ultrasound according to an embodiment of the present disclosure, the reflected signal may be implemented by displaying strength of a plurality of reflected sounds in a time axis in the form of an amplitude size.

In the method of evaluating muscle quality using ultrasound according to an embodiment of the present disclosure, the signal processing step may further include a filtering step of performing, by a filtering unit, filtering processing on the reflected signal and a smoothing processing step of performing, by a smoothing processing unit, smoothing processing on the reflected signal.

In the method of evaluating muscle quality using ultrasound according to an embodiment of the present disclosure, the filtering step may include removing a contact-reflected signal implemented by a reflected sound that is generated at the interface of the ultrasound transceiver and the subject.

In the method of evaluating muscle quality using ultrasound according to an embodiment of the present disclosure, the material may include muscles, a bone, or fat.

In the method of evaluating muscle quality using ultrasound according to an embodiment of the present disclosure, the analysis step further may include steps of extracting an interval between a plurality of peaks included in the reflected signal and calculating the thickness of fat or muscles based on the interval.

In the method of evaluating muscle quality using ultrasound according to an embodiment of the present disclosure, the analysis step may further include a Fourier transform step of performing, by a Fourier transform unit, short time Fourier transform (STFT) processing on the reflected signal and a pimelosis degree calculation step of calculating, by a pimelosis degree calculation unit, a degree of pimelosis of muscles by analyzing the reflected signal on which the STFT processing has been performed.

As an embodiment of the present disclosure, there is provided a computer-readable recording medium on which a program for implementing the aforementioned method has been recorded.

### MODE FOR INVENTION

Hereinafter, embodiments of the present disclosure are described in detail with reference to the accompanying drawings so that a person having ordinary knowledge in the art to which the present disclosure pertains may easily practice the embodiments. However, the present disclosure may be implemented in various different forms and is not limited to the embodiments described herein. In the drawings, in order to clarify a description of the present disclosure, parts not related to the description are omitted, and similar reference numbers are used to refer to similar parts throughout the specification.

Terms used in this specification are briefly described, and embodiments of the present disclosure are then described in detail.

Terms used in the present disclosure are common terms which are now widely used by taking into consideration functions in the present disclosure, but the terms may be changed depending on an intention of a technician skilled in the art, a precedent, or the advent of a new technology. Furthermore, in a specific case, some terms are randomly selected by the applicant. In this case, the meaning of a corresponding term will be described in detail in a descriptive part of a corresponding invention. Accordingly, terms used in the present disclosure should not be simply defined based on their names, but should be defined based on their substantial meanings and contents over the present disclosure.

In the entire specification, unless explicitly described to the contrary, the word “include” and variations, such as “includes” or “including”, will be understood to imply the inclusion of stated components but not the exclusion of any other components. Furthermore, the term “ . . . unit” or “module” described in the specification means a unit for processing at least one function or operation, and the unit may be implemented by hardware or software or a combination of hardware and software. Furthermore, throughout this specification, when it is described that one component is “connected” to the other component, the one component may be “directly connected” to the other component or may be “indirectly connected” to the other component through a third component.

Hereinafter, the present disclosure is described in detail with reference to the accompanying drawings.

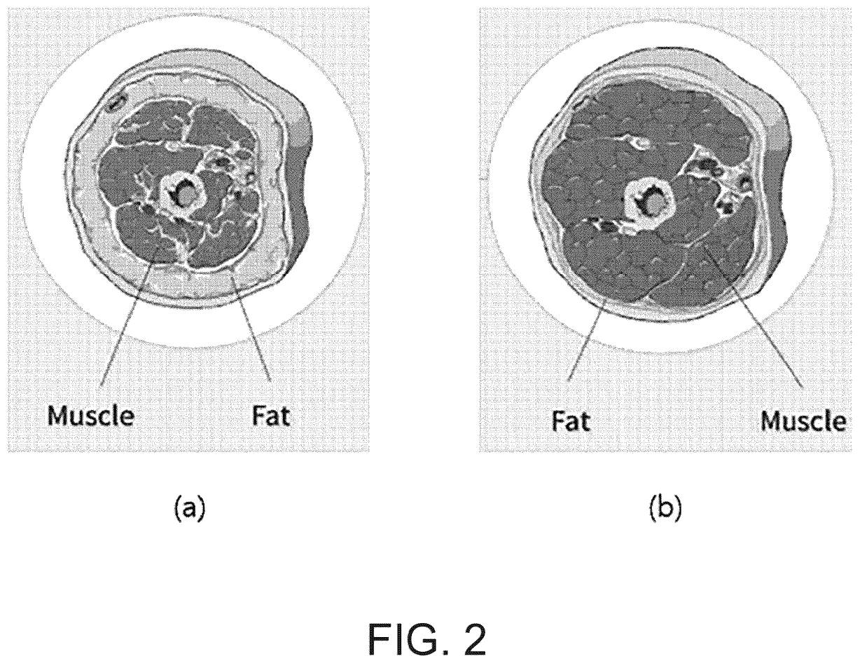

FIG. 2 illustrates a difference between image display methods of a B-mode ultrasonography apparatus and an A-mode ultrasonography apparatus.

Referring to FIG. 2, the B-mode ultrasonography apparatus transforms a reflected signal that is obtained by transmitting and receiving ultrasound to a subject, and outputs the reflected signal in the form of an image, such as that illustrated in FIG. 2(a). In contrast, the A-mode ultrasonography apparatus transforms a reflected signal that is obtained by transmitting and receiving ultrasound to a subject, and may output the reflected signal in the form of a signal as illustrated in FIG. 2(b).

A system for evaluating muscle quality using ultrasound according to an embodiment of the present disclosure evaluates muscle quality by using the output of a reflected sound, which is reflected by a subject and has a signal form, such as that illustrated in FIG. 2(b). Hereinafter, a process of evaluating, by the system according to the present disclosure, muscle quality is described in detail.

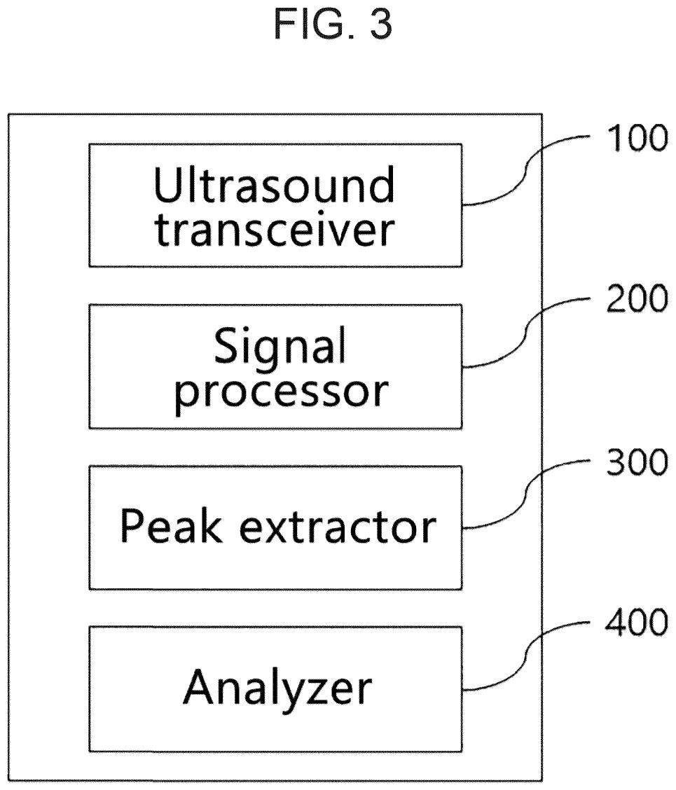

FIG. 3 is a block diagram of the system for evaluating muscle quality using ultrasound according to an embodiment of the present disclosure.

Referring to FIG. 3, the system for evaluating muscle quality using ultrasound according to an embodiment of the present disclosure may include an ultrasound transceiver 100, a signal processor 200, a peak extractor 300, and an analyzer 400.

The ultrasound transceiver 100 may transmit ultrasound to a subject and receive a plurality of reflected sounds that are reflected by the subject. That is, the ultrasound transceiver 100 simultaneously performs the transmission and reception of ultrasound. According to an embodiment, the ultrasound transceiver 100 may be manufactured by using crystal, electrolumines, ceramics, etc.

According to an embodiment, the ultrasound transceiver 100 may be at least one in number. For example, the ultrasound transceiver 100 may be implemented with three A-mode ultrasonic probes. The ultrasound transceiver may transmit and receive ultrasound to and from each of target points at which muscle quality is evaluated, or may be disposed at a specific portion (e.g., the shoulders of a subject) at a predetermined interval and may transmit and receive ultrasound.

The signal processor 200 may implement a reflected signal by using a plurality of reflected sounds. In this case, the reflected signal may be implemented by displaying the strength of the plurality of reflected sounds in the form of an amplitude size in a time axis.

The peak extractor 300 may extract a peak from a reflected signal. In this case, the peak means a case in which the strength of a reflected sound at a specific time point is relatively greater than the strength of a reflected sound at a surrounding time point. The strength f a reflected sound is directly proportional to an impedance difference between materials through which ultrasound passes.

In this case, in the impedance difference between materials that constitute a body, the strength of a reflected sound is the greatest at the interface of muscles and fat. Accordingly, if a point at which the strength of a reflected sound is relatively great, that is, a point at which a peak is generated, in a reflected signal, is extracted, the location of the interface of muscles and fat can be known. According to an embodiment, the peak extractor 300 may be implemented with a Savizky-Golay filter.

The analyzer 400 may calculate the ratio of materials that constitute a subject by using peaks, and may evaluate muscle quality based on the calculated ratio. In this case, the material may include muscles, a bone, or fat, but is not limited thereto and may include all materials that constitute a human body.

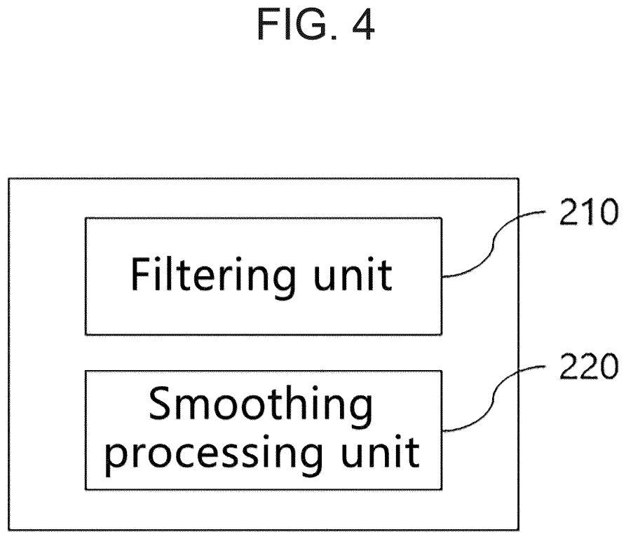

FIG. 4 is a block diagram of the signal processor 200 according to an embodiment of the present disclosure.

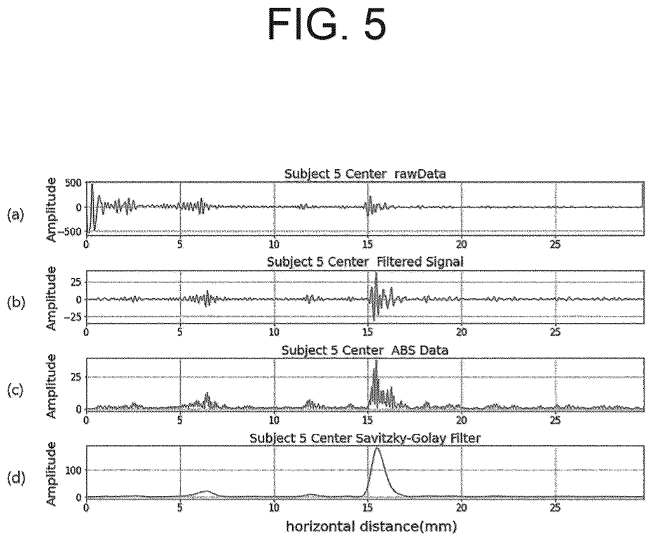

FIG. 5 is a diagram of a signal processing process of the signal processor 200 according to an embodiment of the present disclosure.

Referring to FIGS. 4 and 5, the signal processor 200 according to an embodiment of the present disclosure may further include a filtering unit 210 and a smoothing processing unit 220.

The filtering unit 210 may perform filtering processing on a reflected signal. That is, the filtering unit 210 may remove noise included in the reflected signal or may amplify the signal by performing filtering processing on the reflected signal. In this case, noise may mean all pieces of information except information that is related to the interface of fat and muscles (refer to FIGS. 5(a) to 5(c)). According to an embodiment, the filtering unit 210 may be implemented with a band-pass filter or a squared filter.

According to an embodiment, the filtering unit 210 may remove a contact-reflected signal implemented by a reflected sound that is generated at the interface of the ultrasound transceiver 100 and a subject. The smoothing processing unit 220 may perform smoothing processing on a reflected signal. In this case, the smoothing processing includes extracting only the signal of a major pattern included in the reflected signal, and may mean removing the remaining peak included in the reflected signal (refer to FIG. 5(d)).

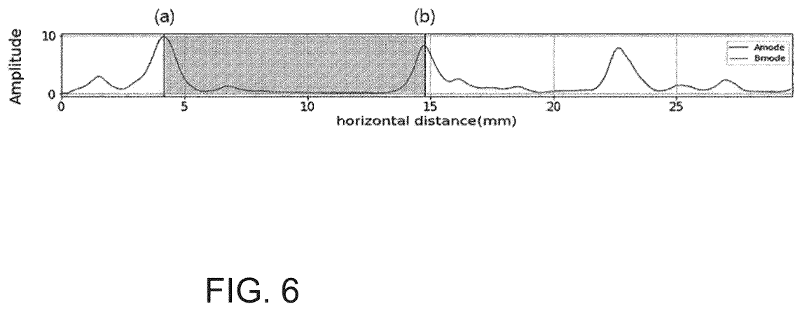

FIG. 6 is a diagram of a muscle quality evaluation process of the analyzer 400 according to an embodiment of the present disclosure.

Referring to FIG. 6, the analyzer 400 according to an embodiment of the present disclosure may extract an interval between a plurality of peaks included in a reflected signal, and may calculate the thickness of fat or muscles based on the interval. For example, the analyzer may find a muscle layer based on an interval between the first peak (a) and the second peak (b), may calculate the thickness of muscles, and may calculate muscle quality (MQ) based on the thickness of the muscles for the entire volume of a subject.

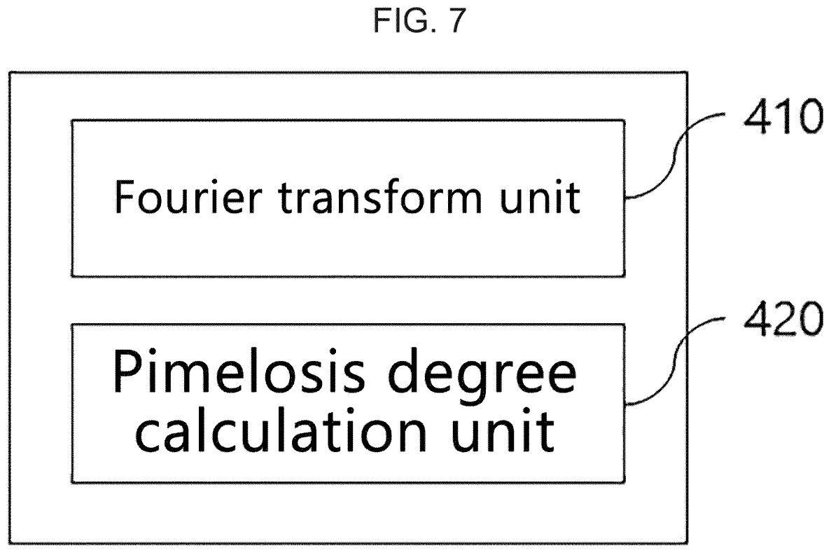

FIG. 7 is a block diagram of the analyzer 400 according to another embodiment of the present disclosure.

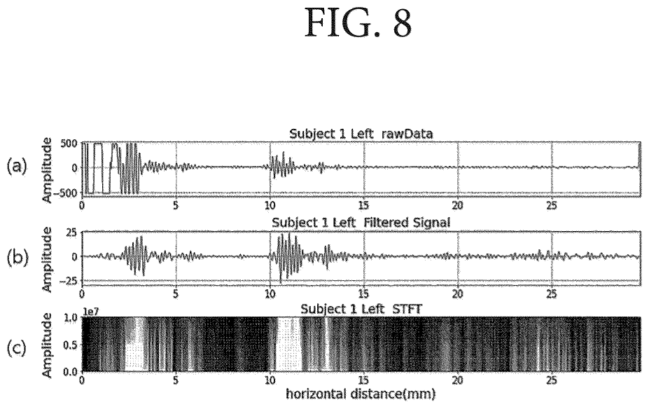

FIG. 8 is a diagram of an STFT process of a Fourier transform unit 410 according to another embodiment of the present disclosure.

Referring to FIG. 7, the analyzer 400 according to another embodiment of the present disclosure may further include the Fourier transform unit 410 and a pimelosis degree calculation unit 420.

FIG. 8(a) is a reflected signal that is implemented by the signal processor 200 by using a reflected sound that is received by the ultrasound transceiver 100. FIG. 8(b) is a reflected signal on which filtering processing has been performed by the filtering unit 210 and from which noise has been removed. FIG. 8(c) is a reflected signal on which short time Fourier transform (STFT) processing has been performed by the Fourier transform unit 410.

Referring to FIG. 8, the Fourier transform unit 410 may perform STET processing on the reflected signal.

In this case, the Fourier transform (FT) means that a signal in a time domain is transformed into a signal in a frequency domain. That is, the analyzer 400 according to the present disclosure may transform a reflected signal in the time domain into a reflected signal in the frequency domain by performing FT processing, and may monitor the reflected signal in the frequency domain. This is for monitoring power of a power spectral density, that is, energy monitoring means, because A-mode ultrasound is used to measure energy of ultrasound that is reflected and returned.

The STFT means that a signal in the time domain is split into signals in a short time unit and FT processing is performed on the signals. The STFT is for monitoring power for each time domain-frequency domain. The analyzer 400 according to the present disclosure may monitor at which time zone a reflected signal is changed for each frequency because ultrasound is time-series data having a time meaning.

The pimelosis degree calculation unit 420 may calculate a degree of pimelosis of muscles by analyzing a reflected signal on which STFT processing has been performed.

According to an embodiment, the pimelosis degree calculation unit 420 may normalize a reflected signal on which STFT processing has been performed, and may evaluate, as muscle quality (MQ), a value that is obtained by dividing the sum of amplitude values of muscles in the normalized reflected signal by the sum of amplitude values of the normalized reflected signal. In this case, the amplitude values of the muscles may be specified based on data that are accumulated through experiments.

In relation to a method according to an embodiment of the present disclosure, the contents of the aforementioned system may be applied. Hereinafter, a description of the same contents as the contents of the aforementioned system in relation to the method is omitted.

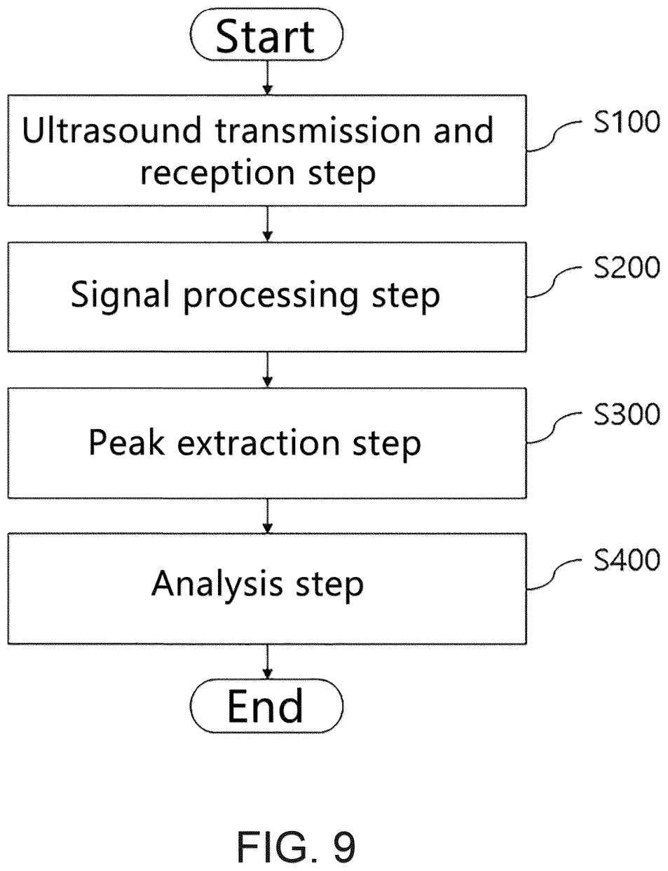

FIG. 9 is a flowchart of a method of evaluating muscle quality using ultrasound according to an embodiment of the present disclosure.

Referring to FIG. 9, the method of evaluating muscle quality using ultrasound according to an embodiment of the present disclosure may include an ultrasound transmission and reception step S100, a signal processing step S200, a peak extraction step S300, and an analysis step S400.

In the ultrasound transmission and reception step S100, the ultrasound transceiver 100 may transmit ultrasound to a subject and receive a plurality of reflected sounds that are reflected by the subject. According to an embodiment, at least one ultrasound transceiver 100 may be implemented.

In the signal processing step S200, the signal processor 200 may implement a reflected signal by using the plurality of reflected sounds. According to an embodiment, the reflected signal may be implemented by displaying the strength of the plurality of reflected sounds in the time axis in the form of an amplitude size.

According to an embodiment, the signal processing step S200 may further include a filtering step S210 of performing, by the filtering unit 210, filtering processing on the reflected signal and a smoothing processing step S220 of performing, by the smoothing processing unit 220, smoothing processing on the reflected signal.

According to an embodiment, in the filtering step S210, a contact-reflected signal implemented by a reflected sound that is generated at the interface of the ultrasound transceiver 100 and the subject may be removed.

In the peak extraction step S300, the peak extractor 300 may extract peaks from the reflected signal.

In the analysis step S400, the analyzer 400 may calculate the ratio of materials that constitute the subject by using the peaks, and may evaluate muscle quality based on the calculated ratio. According to an embodiment, the material may include muscles, a bone, or fat.

According to an embodiment, the analysis step S400 may further include step S411 of extracting an interval between a plurality of peaks that are included in the reflected signal and step S412 of calculating the thickness of fat or muscles based on the interval.

According to another embodiment, the analysis step S400 may further include a Fourier transform step S421 of performing, by the Fourier transform unit 410, short time Fourier transform (STFT) processing on the reflected signal and a pimelosis degree calculation step S422 of calculating, by the pimelosis degree calculation unit 420, a degree of pimelosis of muscles by analyzing the reflected signal on which the STFT processing has been performed.

Meanwhile, the aforementioned method may be written in the form of a computer-executable program, and may be implemented in a general-purpose digital computer that drives the program by using a computer-readable recording medium. Furthermore, the structure of data used in the aforementioned method may be recorded on a computer-readable recording medium through several means. The computer-readable recording medium includes storage media, such as magnetic storage media (e.g., ROM, RAM, a USB, a floppy disk, and a hard disk) and optical recording media (e.g., CD-ROM and a DVD).

The description of the present disclosure is illustrative, and a person having ordinary knowledge in the art to which the present disclosure pertains will understand that the present disclosure may be easily modified in other detailed forms without changing the technical spirit or essential characteristic of the present disclosure. Accordingly, it should be construed that the aforementioned embodiments are only illustrative in all aspects, and are not limitative. For example, components described in the singular form may be carried out in a distributed form. Likewise, components described in a distributed form may also be carried out in a combined form.

The scope of the present disclosure is defined by the appended claims rather than by the detailed description, and all changes or modifications derived from the meanings and scope of the claims and equivalents thereto should be interpreted as being included in the scope of the present disclosure.

1. A system for evaluating muscle quality using ultrasound, the system comprising:
an ultrasound transceiver configured to transmit ultrasound to a subject and to receive a plurality of reflected sounds that are reflected by the subject;
a signal processor configured to implement a reflected signal by using the plurality of reflected sounds;
a peak extractor configured to extract peaks from the reflected signal; and
an analyzer configured to calculate a ratio of materials that constitute the subject by using the peaks and to evaluate muscle quality based on the calculated ratio.

2. The system of claim 1, wherein the ultrasound transceiver is at least one in number.

3. The system of claim 1, wherein the signal processor implements the reflected signal by displaying strength of the plurality of reflected sounds in a time axis in a form of an amplitude size.

4. The system of claim 1, wherein the signal processor further comprises:
a filtering unit configured to perform filtering processing on the reflected signal; and
a smoothing processing unit configured to perform smoothing processing on the reflected signal.

5. The system of claim 4, wherein the filtering unit removes a contact-reflected signal implemented by a reflected sound that is generated at an interface of the ultrasound transceiver and the subject.

6. The system of claim 1, wherein the material comprises muscles, a bone, or fat.

7. The system of claim 1, wherein the analyzer extracts an interval between a plurality of peaks included in the reflected signal and calculates a thickness of fat or muscles based on the interval.

8. The system of claim 1, wherein the analyzer further comprises:
a Fourier transform unit configured to perform short time Fourier transform (STFT) processing on the reflected signal; and
a pimelosis degree calculation unit configured to calculate a degree of pimelosis of muscles by analyzing the reflected signal on which the STFT processing has been performed.

9. A method of evaluating muscle quality using ultrasound, the method comprising:
an ultrasound transmission and reception step of transmitting, by an ultrasound transceiver, ultrasound to a subject and receiving a plurality of reflected sounds that are reflected by the subject;
a signal processing step of implementing, by a signal processor, a reflected signal by using the plurality of reflected sounds;
a peak extraction step of extracting, by a peak extractor, peaks from the reflected signal; and
an analysis step of calculating, by an analyzer, a ratio of materials that constitute the subject by using the peaks and evaluating muscle quality based on the calculated ratio.

10. The method of claim 9, wherein the ultrasound transceiver is at least one in number.

11. The method of claim 9, wherein the reflected signal is implemented by displaying strength of a plurality of reflected sounds in a time axis in a form of an amplitude size.

12. The method of claim 9, wherein the signal processing step further comprises:
a filtering step of performing, by a filtering unit, filtering processing on the reflected signal; and
a smoothing processing step of performing, by a smoothing processing unit, smoothing processing on the reflected signal.

13. The method of claim 12, wherein the filtering step comprises removing a contact-reflected signal implemented by a reflected sound that is generated at an interface of the ultrasound transceiver and the subject.

14. The method of claim 9, wherein the material comprises muscles, a bone, or fat.

15. The method of claim 9, wherein the analysis step further comprises steps of:
extracting an interval between a plurality of peaks included in the reflected signal; and
calculating a thickness of fat or muscles based on the interval.

16. The method of claim 9, wherein the analysis step further comprises:
a Fourier transform step of performing, by a Fourier transform unit, short time Fourier transform (STFT) processing on the reflected signal; and
a pimelosis degree calculation step of calculating, by a pimelosis degree calculation unit, a degree of pimelosis of muscles by analyzing the reflected signal on which the STFT processing has been performed.

17. A computer-readable recording medium on which a program for implementing any one of the methods of claim 9 has been recorded.
